(19) United States
(12) Patent Application Publication
(10) Pub. No.: US 2007/0233274 A1
(43) Pub. Date: Oct. 4, 2007

(54) Artificial Tissue and Process for Producing the Same

(76) Inventor: Hideyuki Miyake, Tokyo (JP)

Correspondence Address: LADAS & PARRY LLP, 224 SOUTH MICHIGAN AVENUE, SUITE 1600, CHICAGO, IL, 60604, US

(21) Appl. No.: 11/547,997

(22) PCT Filed: Apr. 12, 2005
(86) PCT No.: PCT/JP05/07072

§ 371 Date: Oct. 11, 2006

(30) Foreign Application Priority Data
Apr. 12, 2004 (JP) 2004-117175

(51) Int. Cl. A61F 2/02

(52) U.S. Cl. 623/23.72

(57) ABSTRACT

A main object of the present invention is to provide an artificial tissue capable of transporting the nutrition necessary for maintaining the activity of the cells or tissues. To achieve the object, the present invention provides an artificial tissue including a blood vessel-containing tissue layer having at least two adjacent blood vessels and a cell disposed between the blood vessels, characterized in that an interval between the two adjacent blood vessels in the blood vessel-containing tissue layer is formed by a nutrition supplyable distance which does not cause a necrosis of the cell.

### TECHNICAL FIELD

The present invention relates to an artificial tissue used in the field of the regenerative medicine, or the like.

### BACKGROUND ART

At present, cell cultures of various animals and plants are performed, and also new cell culture methods are in development. The technologies of the cell culture are utilized, such as to elucidate the biochemical phenomena and natures of cells and to produce useful substances. Furthermore, with cultured cells, an attempt to investigate the physiological activity and toxicity of artificially synthesized medical is under way. Moreover, in the field of the medicine and others, artificial production of tissues and organs has been attempted by re-organizing such as cells, proteins, glucides, or lipids of living bodies by the technique of the cell engineering, or the like.

Here, since the common animal cells perish without supply of the nutrition, or the like, in the case of using cultured cells as the artificial tissues, or the like, it is necessary to provide the capillary vessels in the artificial tissues and the blood for passing through therein for supplying such as the oxygen or nutrition, and carrying out the wastes. Conventionally, for example, artificial formation of the capillary vessels has been attempted by the techniques of the non-patent documents 1 to 3, however, in either case, only the vessel-like tissues (capillaries) are formed in disorder so that it has been difficult to form capillary cells capable of providing a necessary amount of the blood to a desired position for maintaining the function of the artificial tissues. Moreover, as shown in the non-patent documents 4 and 5, although the method for forming a blood vessel with an artificial material has been studied, since it is difficult to form a thin blood vessel, it cannot be utilized for such an artificial tissue.

On the other hand, the present inventors have proposed a method of culturing cells in a pattern by changing the surface of a layer having cell adhesive properties or cell adhesion-inhibiting properties by the function of a photocatalyst accompanied by the irradiation with energy for forming a pattern comprising a cell adhesive portion and a cell adhesion-inhibiting portion and highly accurately adhering the cells only to the cell adhesive portion. According to the patterning method, the cells are stimulated at the boundary of the cell adhesive portion and the cell adhesion-inhibiting portion so that the cells adhered in a pattern can be aligned or the morphological change to the stretching state can be promoted strongly as a result. Moreover, since the cells can be cultured easily in a purposed pattern, the vascular tissue formation can be facilitated along a desired pattern, and furthermore, a thin blood vessel can be formed. However, an artificial tissue utilizing the blood vessel has not been invented.

[Non-patent document 1] D. E. Ingber, et al., The Journal of Cell Biology (1989) p. 317

[Non-patent document 2] B. J. Spargo, et al., Proceedings of the National Academy of Sciences of the United States of America (1994) p. 11070

[Non-patent document 3] R. Auerbach et al., Clinical Chemistry (2003) p. 32

[Non-patent document 4] C. B. Weinberg, et al., Science (1986) p. 397

[Non-patent document 5] N. L′. Heureux, et al., The FASEB Journal (1998) vol. 12 p. 47

### DISCLOSURE OF THE INVENTION

### Problem to be Solved by the Invention

For the above-mentioned reasons, supply of the necessary oxygen and nutrition, and discharge of the wastes are indispensable for constructing the artificial tissues and organs to keep the functions thereof so that an artificial tissue accompanying such a substance conveyance mechanism is desired.

### MEANS FOR SOLVING THE PROBLEM

The present invention provides an artificial tissue including a blood vessel-containing tissue layer having at least two adjacent blood vessels and a cell disposed between the blood vessels, characterized in that an interval between the two adjacent blood vessels in the blood vessel-containing tissue layer is formed by a nutrition supplyable distance which does not cause a necrosis of the cell.

According to the present invention, since two adjacent blood vessels are formed in a nutrition supplyable distance which does not cause the necrosis of the cell in the above-mentioned blood vessel-containing tissue, the cell in the artificial tissue can have the supply of such as the oxygen or the nutrition through the blood vessels. Therefore, various ones can be used as the above-mentioned cell so that an artificial tissue to be used for example as an organ can be provided.

In the above-mentioned invention, the above-mentioned blood vessel-containing tissue layer can be laminated by at least two or more layers. Thereby, the blood vessels and the above-mentioned cell can be disposed three-dimensionally so that a further complicated artificial tissue can be provided.

The present invention further provides a process for producing an artificial tissue comprising a blood vessel-containing tissue layer having at least two adjacent blood vessels and a cell disposed between the blood vessels, characterized by comprising: a blood vessel disposing process of disposing the two adjacent blood vessels with a nutrition supplyable distance which does not cause a necrosis of the cell, and a cell contacting process of contacting a cell containing layer containing the cell and the blood vessels.

According to the present invention, since two adjacent blood vessels are disposed in the above-mentioned nutrition supplyable distance in the above-mentioned blood vessel disposing process, nutrition can be supplied to the cell contacted by the cell contacting process. Therefore, various artificial tissues can be produced without necrosis caused, or the like of the cell in the formed artificial tissue.

In the above-mentioned invention, the above-mentioned blood vessel disposing process may be a process of forming at least two or more of the above-mentioned blood vessels on a vascular cell culture substrate so that the blood vessels have a distance wider than the above-mentioned nutrition supplyable distance, and removing a part of the above-mentioned vascular cell culture substrate disposed between the above-mentioned blood vessels. Alternatively, the above-mentioned blood vessels disposing process may be a process of forming at least two or more of the above-mentioned blood vessel in a state with the above-mentioned vascular cell culture substrate stretched on a vascular cell culture substrate having stretching properties, and shortening the above-mentioned vascular cell culture substrate so as to shorten the distance between the above-mentioned blood vessels. Here, at the time of forming a plurality of the blood vessels on the vascular cell culture substrate, if the distance between the blood vessel forming cells for forming the adjacent blood vessels is short, the adjacent blood vessel forming cells are contacted via the pseudopods, or the like. As a result, the vascular cells are stimulated so as to generate the adhesion between the adjacent blood vessels at the time of forming a vascular tissue so that a blood vessel of a desired shape cannot be formed.

Therefore, in general, a plurality of blood vessels cannot be formed on one vascular cell culture substrate with the above-mentioned nutrition supplyable distance.

Then, according to the present invention, it is preferable that the above-mentioned blood vessel disposing process is a process of forming blood vessels with an interval of the above-mentioned nutrition supplyable distance or wider, and thereafter disposing the blood vessels so as to have the above-mentioned nutrition supplyable distance between the adjacent blood vessels as mentioned above.

### EFFECT OF THE INVENTION

According to the present invention, the advantages of providing an artificial tissue without causing a necrosis, or the like of the cells in the tissue and providing an artificial tissue to be used as, for example, an organ by use of various ones as the above-mentioned cells can be achieved.

### BRIEF DESCRIPTION OF THE DRAWINGS

FIG. 1 is a plan view showing an example of a blood vessel-containing tissue layer of the present invention.

FIGS. 2A and 2B are each a schematic cross-sectional view for explaining a blood vessel-containing tissue layer of the present invention.

FIG. 3 is a schematic sectional view showing an example of the photocatalyst-containing layer side substrate used in the present invention.

FIG. 4 is a schematic sectional view showing another example of the photocatalyst-containing layer side substrate used in the present invention.

FIG. 5 is a schematic sectional view showing another example of the photocatalyst-containing layer side substrate used in the present invention.

FIGS. 6A and 6B are an explanatory diagram showing an example of a method for forming a cell adhesive portion and a cell adhesion-inhibiting portion of a vascular cell culture substrate of the present invention.

### EXPLANATION OF REFERENCES

1 blood vessel
2 cell
3 blood vessel-containing tissue layer

### BEST MODE FOR CARRYING OUT THE INVENTION

The present invention relates to an artificial tissue used in the field of the regenerative medicine, or the like, a process for producing the same. Hereinafter, each will be explained in detail.

A. Artificial Tissue

First, the artificial tissue of the present invention will be explained. The artificial tissue of the present invention is produced by artificial re-organization of cells having various functions taken out from a living body by a cell engineering technique, or the like. Moreover, it is an artificial tissue including a blood vessel-containing tissue layer having at least two adjacent blood vessels and a cell disposed between the above-mentioned blood vessels, wherein the interval between the above-mentioned two adjacent blood vessels in the above-mentioned blood vessel-containing tissue layer is formed by a nutrition supplyable distance which does not cause a necrosis of the above-mentioned cell.

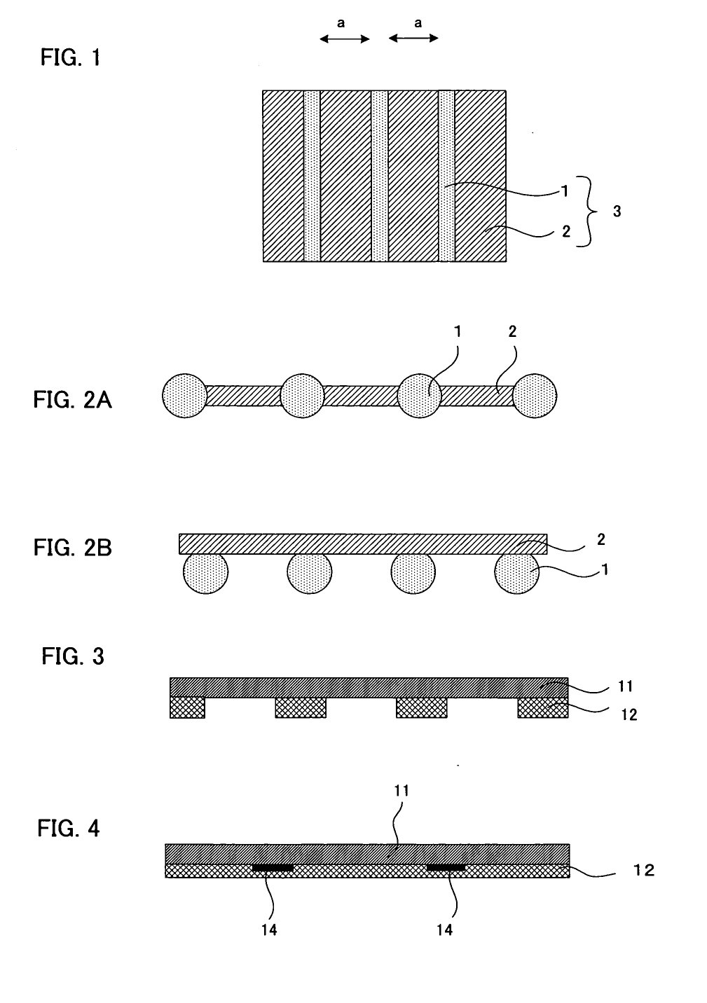

For example as shown in FIG. 1, the artificial tissue of the present invention includes a blood vessel-containing tissue layer 3 with the distance “a” between at least two adjacent blood vessels 1 provided as a distance which does not cause a necrosis of a cell 2 disposed between the blood vessels 1.

According to the present invention, the above-mentioned blood vessels can play a role of supplying such as the oxygen or nutrition to the above-mentioned cell, and taking out the wastes discharged from the cell, or the like in the above-mentioned blood vessel-containing tissue layer. Moreover, since the distance between the above-mentioned adjacent blood vessels is provided as the above-mentioned nutrition supplyable distance, the all cells in the artificial tissue can have the supply of the oxygen, nutrition, or the like by the blood vessels. Therefore, by using various cells as the above-mentioned cell, an artificial tissue to be used such as for an organ can be provided. Here, the nutrition in the present invention refers to the substances necessary for maintaining the activity of a living body, and furthermore, necessary for existence of the cells, including such as glucides, lipids, proteins, and furthermore, oxygen to react with these substances. In general, a medium for conveying the nutrition in a living body is the blood, and a culture solution (culture medium) in a cell culture system.

Hereinafter, the artificial tissue of the present invention will be explained in detail for each configuration.

1. Blood Vessel-Containing Tissue Layer

First, the blood vessel-containing tissue layer in the artificial tissue of the present invention will be explained. The blood vessel-containing tissue layer in the artificial tissue of the present invention is not particularly limited as long as it has at least two adjacent blood vessels and the cell disposed between the above-mentioned blood vessels, with the interval between the two adjacent blood vessels formed in a nutrition supplyable distance which does not cause a necrosis of the above-mentioned cell.

Here, the nutrition supplyable distance which does not cause a necrosis of the above-mentioned cell is a distance capable of supplying such as the oxygen or nutrition form the blood vessels to the all cells disposed between the two adjacent blood vessels. The nutrition supplyable distance differs significantly depending on the nutrition supplyability of the blood vessels, the kind of the cell, or the like so that it can be selected optionally according to the purposed artificial tissue. Here, as the range capable of supplying the nutrition from one blood vessel, it is in general by a radius from the center of the blood vessel of 600 μm or less, in particular, 300 μm or less. Although the lower limit is not particularly limited, it is preferably a distance which does not cause a zygosis of the adjacent blood vessels, and it can be for example 10 μm or more, in particular, 30 μm or more. Then, in the present invention, the above-mentioned nutrition supplyable distance can be set by a distance two times of the above-mentioned nutrition supplyable range from one blood vessel.

Here, in the present invention, at least two or more blood vessels can be disposed like substantially parallel lines. The “substantially parallel lines” means a state without intersection of two lines in a certain region so that for example zigzag lines such as with the lines present without intersection can also be included. The distance between the above-mentioned lines is provided as the nutrition supplyable distance. Moreover, at the time, the blood vessels can be intersected or branches in for example a mesh structure. In this case, the distance between the blood vessels in a portion without intersection or branching of the blood vessels is the above-mentioned nutrition supplyable distance.

Moreover, the shape of the above-mentioned blood vessel-containing tissue is not particularly limited so that it can be selected optionally according to the shape of the purposed artificial tissue, or the like. Here, in the blood vessel-containing tissue, for example as shown in FIG. 2A, the cell may be disposed between the adjacent blood vessels 1, or as shown in FIG. 2B, a sheet-like cell 2 may be disposed on the blood vessels 1.

Such a blood vessel-containing tissue layer can be formed by for example culturing the cell between the blood vessels disposed by the nutrition supplyable distance, or attaching the blood vessels disposed by the nutrition supplyable distance, and a cell layer cultured independently from the blood vessels.

Hereinafter, the blood vessels and the cell used for the above-mentioned blood vessel-containing tissue layer will be explained in detail.

<Blood Vessel>

First, the blood vessel used in the present invention will be explained. The blood vessel used in the present invention is not particularly limited as long as it can supply such as the oxygen or nutrition to the cell to be described later, transport the wastes produced by the other cells between the blood vessels, or the like.

Such a blood vessel can be formed by culturing in a pattern the blood vessel forming cell to be cultured for organizing a blood vessel, and adding a growth factor for facilitating the vascularization of the blood vessel forming cells, or the like. Such blood vessel forming cell for organizing a blood vessel refers to vascular endothelial cells, pericytes, smooth muscle cells, endothelial precursor cells and smooth muscle precursor cells derived from organisms, particularly men and animals. Particularly, it refers to vascular endothelial cells etc. Plural kinds of cells can be co-cultured such as co-culture of vascular endothelial cells and pericytes or co-culture of endothelial cells and smooth muscle cells.

Usually, a blood vessel is obtained by forming the vascular cells in an objective pattern on the cell adhesion portion, and then, adding, to a medium, growth factors such as bFGF and VEGF promoting vascularization of vascular cells. It is estimated that, by stimulation from the growth factors, proliferation of the vascular cells is terminated and differentiated so as to be blood vessels. As the medium for vascularization of vascular cells adhered in a confluent state to the cell adhesion portion, not only a liquid medium containing the growth factor, but also a gelled medium containing the above-described growth factor or a combination of gelled and liquid mediums containing the growth factor can be used. As the gelled medium, such as collagen, fibrin gel, Matrigel (trade name) or synthetic peptide hydrogel can be used.

Here, when a plurality of blood vessels are formed by patterning on one vascular cell culture substrate, in the case the blood vessel forming cells for forming the adjacent blood vessels are provided adjacently, the blood vessel forming cells are contacted via the pseudopods, or the like so that as a result the adjacent blood vessels are adhered, deformed, or the like so as to fail to form a blood vessel while maintaining a purposed shape. On the other hand, if the distance between the blood vessels is prolonged to the extent that the adhesion, deformation, or the like of the blood vessels can be prevented, it exceeds the distance capable of supplying such as the nutrition from each blood vessel to the surrounding cells so that it has been difficult to supply the nutrition to the surrounding cells.

Then, in the present invention, the blood vessels can be used after forming on the vascular cell culture substrate with an interval of the nutrition supplyable distance or more provided, by moving the formed blood vessels so as to be disposed with the nutrition supplyable distance or more, or the like. Moreover, the above-mentioned blood vessels can be used by for example forming on the vascular cell culture substrate with an interval of the nutrition supplyable distance or more, and removing a part of the vascular cell culture substrate between the adjacent blood vessels so as to be disposed by the nutrition supplyable distance. Furthermore, they can be used by preliminarily stretching a vascular cell culture substrate having stretching properties, forming on the vascular cell culture substrate with an interval of the nutrition supplyable distance or more provided on the vascular cell culture substrate, and shortening the vascular cell culture substrate so as to be disposed by the nutrition supplyable distance.

As to the method for culturing the above-mentioned blood vessel forming cells in a pattern, it is preferable to use for example a method of culturing blood vessel forming cells in a pattern by forming on a base material a cell adhesion layer containing a cell adhesive material having adhesive properties with the blood vessel forming cell, to be decomposed or denatured by the function of a photocatalyst accompanied by the irradiation with energy, or a cell adhesion-inhibiting layer containing cell adhesion-inhibiting properties of inhibiting adhesion with the blood vessel forming cell, to be decomposed or denatured by the function of a photocatalyst accompanied by the irradiation with energy, and providing the function of the photocatalyst accompanied by the irradiation with energy in a pattern for providing the cell adhesive properties only in the pattern for culturing the blood vessel forming cell.

According to the method, the region other than the region for culturing the blood vessel forming cell can be provided with the cell adhesion-inhibiting properties so that the blood vessel forming cells can be formed easily in a purposed pattern. Furthermore, the cell morphological change, or the like for forming the tissue by the blood vessel forming cells receiving the stimuli can be generated easily between the region having the cell adhesive properties and the region having the cell adhesion-inhibiting properties so that the blood vessel can be formed easily.

To form the blood vessels, using the cell adhesion portion having the cell adhesive properties, it is effective to apply shearing stress in uniaxial direction in the same direction as the line pattern of the cell adhesion portion. The adhered form of the vascular cells can thereby become long and thin spindle-shaped, and the respective vascular cells can adhere to one another in such a state that they seem aligned in the uniaxial direction described above. To form the blood vessels, it is important that the vascular cells are adhered in a confluent state such that the vascular cells are adhered in a thin and long form and the vascular cells are directed to the same direction. The method for applying shear stress in the uniaxial direction includes: a method in which the vascular cells are cultured by placing a culture dish on a shaker or a shaking apparatus; and a method in which the vascular cells are cultured while streaming culture liquid in one direction. To form a blood vessel of over 5000 μm in width, shearing stress in uniaxial direction is essential.

Hereinafter, the vascular cell culture substrate having the cell adhesion layer or the cell adhesion-inhibiting layer for culturing the blood vessel forming cell utilizing the function of the photocatalyst accompanied by the irradiation with energy will be explained in detail.

(Vascular Cell Culture Substrate)

As the vascular cell culture substrate having a cell adhesion layer or a cell adhesion-inhibiting layer to have the adhesive properties change with respect to the cell by the function of the photocatalyst accompanied by the irradiation with energy, used in the present invention, for example, the following two embodiments can be presented. Each embodiment will be explained in detail.

### (1) First Embodiment

The first embodiment is a vascular cell culture substrate wherein: a cell adhesion layer, containing a cell adhesive material having at least adhesive properties to a blood vessel forming cell on the base material and is decomposed or denatured by the action of a photocatalyst upon irradiation with energy, is formed; and in the cell adhesion-inhibiting portion, the cell adhesive material is decomposed or denatured by the action of a photocatalyst upon irradiation with energy.

In this embodiment, for example, by arranging the cell adhesion layer formed on the base material and a photocatalyst-containing layer side substrate comprising a photocatalyst-containing layer containing a photocatalyst so as to be opposite to each other and irradiating with energy in a pattern of a cell adhesion-inhibiting portion to be formed, the cell adhesive material in the cell adhesion layer will be decomposed or denatured by the action of the photocatalyst in the photocatalyst-containing layer to form a cell adhesion-inhibiting portion.

In this embodiment, there is an advantage that, when blood vessel forming cells are adhered to the cell adhesion portion on the cell culture patterning substrate to manufacture blood vessels, by irradiating the cell adhesion-inhibiting portion forming region with energy by using the photocatalyst-containing layer, the blood vessel forming cells adhered to the cell adhesion-inhibiting portion can be removed by the action of the photocatalyst, and thus the blood vessel forming cells cultured in a highly precise pattern can be maintained.

In this embodiment, the surface distance of the adjacent cell adhesion portions, that is the surface distance of the cell adhesion-inhibiting portions is usually about 200 μm to 600 μm, particularly about 300 μm to 500 μm. In this range, the blood vessel forming cells can be prevented from contacting with each other via pseudopods between the adjacent cell adhesion portions.

The shape of the cell adhesion portion is not particularly limited insofar as it is formed in a line form.

The shape is selected suitably depending on the shape of an objective blood vessel. Usually, the line width of the cell adhesion portion shall be about 10 μm to 5000 μm, especially 20 μm to 100 μm, particularly 40 μm to 60 μm. A line width of less than 10 μm is not preferable because adhesion of vascular cells is made difficult. A line width of over 5000 μm, on the other hand, is not preferable either because almost all vascular cells will be adhered to the cell adhesion portion in a spread state, thus making the cultured vascular cells hardly formable in the form of a blood vessel.

In the present embodiment, particularly the cell adhesion portion preferably has a cell adhesion auxiliary portion in order to form an excellent blood vessel. The cell adhesion auxiliary portion refers to a region not having adhesive properties to vascular cells, which are formed in a fine pattern on the cell adhesion portion. The cell adhesion auxiliary portion is formed in such a fine pattern to an extent that, when vascular cells are adhered onto the cell adhesion portion, binding of the vascular cells to one another in the cell adhesion portion is not prevented. That is, to an extent that the cells can be bound to one another even on the cell adhesion auxiliary portion.

Generally, when vascular cells are adhered to a cell adhesion portion and cultured to form a tissue, the vascular cells are gradually arranged from the outside toward inside of a cell adhesion portion. For forming a tissue, individual vascular cells should be changed morphologically and arranged, and this morphological change of the vascular cell also gradually occurs from the edge part toward center part of the cell adhesion portion. Accordingly, when the width of the cell adhesion portion is large, a tissue may not be formed in the center part of the cell adhesion portion because of insufficient arrangement of the vascular cells, or the vascular cells may fail to adhere to the center part of the cell adhesion portion. Moreover, the morphological change of the vascular cells in the center part of the cell adhesion portion may be insufficient. Therefore, by forming the cell adhesion auxiliary portion, the vascular cells can be arranged or morphologically changed from the edge part of the cell adhesion auxiliary portion. Thereby, the vascular cells can be cultured without generating such as defects or inferior morphological change. Moreover, the cell adhesion auxiliary portion is formed such that vascular cells adjacent to one another via the cell adhesion auxiliary portion are not prevented from being adhered to one another. Thus, the width of the finally cultured vascular cells can be the same as the width of the cell adhesion portion.

The cell adhesion auxiliary portion is formed preferably in a line form in the cell adhesion portion. The shape of the line is not particularly limited and can be in the form of, for example, a straight line, a curved line, a dotted line or a broken line. The line width of the cell adhesion auxiliary portion is preferably in the range of 0.5 μm to 10 μm, more preferably 1 μm to 5 μm. The width larger than the above range is not preferable because the vascular cells adjacent to one another via the cell adhesion auxiliary portion will hardly interact with one another on the cell adhesion auxiliary portion. When the width is smaller than the above range, on the other hand, the cell adhesion auxiliary portion will be hardly formed by pattern forming techniques of the present embodiment.

The cell adhesion auxiliary portion may be formed to have a convexoconcave pattern (for example, zigzag) in plane. The term “in plane” refers to the surface of a base material or a surface analogous thereto. The average distance from the edge part of the concave portion to the edge part of the convex portion, of the convexoconcave pattern, may be such a distance that when vascular cells are adhered to the cell adhesion portion, the vascular cells are aligned in the same direction as the line direction of the cell adhesion portion, and the average distance is particularly preferably in the range of 0.5 μm to 30 μm. The average distance from the edge part of the concave portion to the edge part of the convex portion of the convexoconcave pattern is determined by calculating the average of measured distances from the lowermost bottom to the uppermost top of each concavoconvex, within the range of 200 μm of the edge portion of the cell adhesion auxiliary portion. Formation of the cell adhesion auxiliary portion is same as the method for forming a cell adhesion-inhibiting portion.

Hereinafter, the cell adhesion layer and the photocatalyst-containing layer side substrate used in the present embodiment, and the method of forming the cell adhesion-inhibiting portion using the photocatalyst-containing layer side substrate will be explained.

a. Cell Adhesion Layer

Now, the cell adhesion layer used in this embodiment is described. The cell adhesion layer used in this embodiment is a layer having at least a cell adhesive material having adhesive properties to a blood vessel forming cell. Generally, a layer used as a layer having adhesive properties to blood vessel forming cells can be used.

The type etc. of the cell adhesive material contained in the cell adhesion layer in this embodiment are not particularly limited insofar as the material has adhesive properties to a blood vessel forming cell and is decomposed or denatured by the action of the photocatalyst upon irradiation with energy. Here, “having adhesive properties to a blood vessel forming cell” means being good in adhesion to a blood vessel forming cell. For instance, when the adhesive properties to a blood vessel forming cell differ depending on the kind of the blood vessel forming cell, it means to be good in the adhesion with the target blood vessel forming cell.

The cell adhesive material used in the present embodiment has such adhesive properties to a blood vessel forming cell. Those losing the adhesive properties to a blood vessel forming cell or those changed into ones having the cell adhesion-inhibiting properties of inhibiting adhesion to blood vessel forming cells, by being decomposed or denatured by the action of the photocatalyst upon irradiation with energy, are used.

As such materials having the adhesive properties to a blood vessel forming cell, there are two kinds. One is being materials having the adhesive properties to a blood vessel forming cell owing to physicochemical characteristics and the other being materials having the adhesive properties to a blood vessel forming cell owing to biochemical characteristics.

As physicochemical factors that determine the adhesive properties to a blood vessel forming cell of the materials having the adhesive properties to a blood vessel forming cell owing to the physicochemical characteristics, the surface free energy, the electrostatic interaction and the like can be cited. For instance, when the adhesive properties to a blood vessel forming cell is determined by the surface free energy of the material, if the material has the surface free energy in a predetermined range, the adhesive properties between the blood vessel forming cell and the material becomes good. If it deviates from the predetermined range the adhesive properties between the blood vessel forming cell and material is deteriorated. As such changes of the adhesive properties to a cell due to the surface free energy, experimental results shown in Data, for instance, CMC Publishing Co., Ltd. “Biomaterial no Saisentan”, Yoshito IKADA (editor), p. 109, lower part are known. As materials having the adhesive properties to a blood vessel forming cell owing to such a factor, for instance, hydrophilic polystyrene, and poly (N-isopropyl acrylamide) can be cited. When such a material is used, by the action of the photocatalyst upon irradiation with energy, for instance, a functional group on a surface of the material is substituted, decomposed or the like to cause a change in the surface free energy, resulting in one that does not have the adhesive properties to a blood vessel forming cell or one that has the cell adhesion-inhibiting properties.

When the adhesive properties between blood vessel forming cell and a material is determined owing to such as the electrostatic interaction, the adhesive properties to a blood vessel forming cell are determined by such as an amount of positive electric charges that the material has. As materials having the adhesive properties to a blood vessel forming cell owing to such electrostatic interaction, basic polymers such as polylysine; basic compounds such as aminopropyltriethoxysilane, N-(2-aminoethyl)-3-aminopropyltrimethoxysilane; and condensates and the like including these can be cited. When such materials are used, by the action of the photocatalyst upon irradiation with energy, the above-mentioned materials are decomposed or denatured. Thereby, for instance, an amount of positive electric charges present on a surface can be altered, resulting in one that does not have the adhesive properties to a blood vessel forming cell or one that has the cell adhesion-inhibiting properties.

As materials having the adhesive properties to a blood vessel forming cell owing to the biological characteristics, ones that are good in the adhesive properties with particular blood vessel forming cell or ones that are good in the adhesive properties with many blood vessel forming cells can be cited. Specifically, fibronectin, laminin, tenascin, vitronectin, RGD (arginine-glycine-asparagine acid) sequence containing peptide, YIGSR (tyrosine-isoleucine-glycine-serine-arginine) sequence containing peptide, collagen, atelocollagen, gelatin and mixture thereof, such as matrigel, can be cited. When such materials are used, by the action of the photocatalyst upon irradiation with energy, for instance, a structure of the material is partially destroyed, or a principal chain is destroyed, resulting in one that does not have the adhesive properties to a blood vessel forming cell or one that has the cell adhesion-inhibiting properties.

Such a cell adhesive material, though it differs depending on the kind of the materials and the like, is comprised in the cell adhesion layer normally in the range of 0.01% by weight to 95% by weight, and preferably in the range of 1% by weight to 10% by weight. Thereby, a region that contains the cell adhesive material can be made a region good in the adhesive properties to a blood vessel forming cell.

In this embodiment, not only the cell adhesive material but also a binder etc. for improving such as strength or resistance may be contained as necessary in the cell adhesion layer. In the present embodiment, particularly as the binder, a material that, at least after the energy irradiation, has the cell adhesion-inhibiting properties of inhibiting adhesion to the blood vessel forming cell is preferably used. This is because the adhesion between the blood vessel forming cell and the cell adhesion-inhibiting portion, which is a region irradiated with energy, can thereby be reduced. As such a material, for example, one that has the cell adhesion-inhibiting properties prior to the energy irradiation or one that obtains the cell adhesion-inhibiting properties by the action of the photocatalyst upon irradiation with energy may be used.

In the present embodiment, a material that becomes to have the cell adhesion-inhibiting properties, particularly by the action of the photocatalyst upon irradiation with energy, is preferably used as a binder. Thereby, in a region prior to the energy irradiation, the adhesiveness between the cell adhesive material and the blood vessel forming cell is not inhibited, and only a region where energy is irradiated can be lowered in the adhesive properties to a blood vessel forming cell.

As materials that can be used as such a binder, for instance, ones in which a principal skeleton has such a high bond energy, that cannot be decomposed by the photo-excitation of the photocatalyst, and has an organic substituent which can be decomposed by an action of the photocatalyst are preferably used. For instance, (1) organopolysiloxane that exhibits large strength by hydrolyzing or polycondensating chloro- or alkoxysilane or the like owing to a sol-gel reaction and the like, and (2) organopolysiloxane and the like in which reactive silicones excellent in the water repellency or oil repellency are crosslinked can be cited.

In the case of the (1), it is preferable to be organopolysiloxanes that are hydrolysis condensates or cohydrolysis condensates of at least one kind of silicon compounds expressed by a general formula:
YₙSiX₍₄₋ₙ₎
(Here, Y denotes an alkyl group, fluoroalkyl group, vinyl group, amino group, phenyl group, epoxy group or organic group containing the above, and X denotes an alkoxyl group, acetyl group or halogen. “n” is an integer of 0 to 3). The number of carbons of the organic group expressed with Y is preferably in the range of 1 to 20, and the alkoxy group shown with X is preferably a methoxy group, ethoxy group, propoxy group or butoxy group.

As the reactive silicone according to the (2), compounds having a skeleton expressed by a general formula below can be cited.

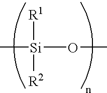

In the above general formula, n denotes an integer of 2 or more, R¹ and R² each represents a substituted or nonsubstituted alkyl group, alkenyl group, aryl group or cyanoalkyl group having 1 to 20 carbons, and a vinyl, phenyl and halogenated phenyl occupy 40% or less by mole ratio to a total mole. Furthermore, one in which R¹ and R² is a methyl group is preferable because the surface energy is the lowest, and a methyl group is preferably contained 60% or more by mole ratio. Still furthermore, a chain terminal or side chain has at least one or more reactive group such as a hydroxyl group in a molecular chain. When the material such as mentioned above is used, by the action of the photocatalyst upon irradiation with energy, a surface of an energy-irradiated region can be made high in the hydrophilicity. Thereby, the adhesion with blood vessel forming cell is inhibited, and the region where energy is irradiated can be made into a region on which the blood vessel forming cell does not adhere.

Together with the organopolysiloxanes, a stable organo silicium compound that does not cause a crosslinking reaction, such as dimethylpolysiloxanes, may be blended with a binder.

When the above-mentioned material is used as the cell adhesion-inhibiting material, the contact angle thereof with water is preferably in the range of 15° to 120°, more preferably 20° to 100° before the material is irradiated with energy. According to this, the adhesion of the cell adhesive material to the blood vessel forming cell is not inhibited.

In the case of irradiating this cell adhesion-inhibiting material with energy, it is preferred that the contact angle thereof with water becomes 10° or less. This range makes it possible to render the material having a high hydrophilicity and low adhesive properties to a blood vessel forming cell.

The contact angle with water referred to herein is a result obtained by using a contact angle measuring device (CA-Z model, manufactured by Kyowa Interface Science Co., Ltd.) to measure the contact angle of the material with water or a liquid having a contact angle equivalent to that of water (after 30 seconds from the time when droplets of the liquid are dropped down from its micro syringe), or a value obtained from a graph prepared from the result.

In the present embodiment, a decomposition substance or the like that causes such as a change in the wettability of a region where energy is irradiated, thereby lowers the adhesive properties to a blood vessel forming cell or that aides such a change may be contained.

As such decomposition substances, for instance, surfactants that are decomposed and the like, by the action of the photocatalyst upon irradiation with energy, to be hydrophilic and the like to result in lowering the adhesive properties to a blood vessel forming cell can be cited.

Specifically, nonionic surfactants: hydrocarbon based such as respective series of NIKKOL BL, BC, BO, and BB manufactured by Nikko Chemicals Co., Ltd.; and silicone based such as ZONYL FSN and FSO manufacture by Du Pont Kabushiki Kaisha, Surflon S-141 and 145 manufactured by ASAHI GLASS CO., LTD., Megaface F-141 and 144 manufactured by DAINIPPON INK AND CHEMICALS, Inc., FTERGENT F-200 and F-251 manufactured by NEOS, UNIDYNE DS-401 and 402 manufactured by DAIKIN INDUSTRIES, Ltd., and FluoradFC-170 and 176 manufactured by 3M can be cited. Cationic surfactants, anionic surfactants and amphoteric surfactants also can be used.

Other than the surfactants, oligomers and polymers such as polyvinyl alcohol, unsaturated polyester, acrylic resin, polyethylene, diallyl phthalate, ethylene propylene diene monomer, epoxy resin, phenol resin, polyurethane, melamine resin, polycarbonate, polyvinyl chloride, polyamide, polyimide, styrene-butadiene rubber, chloroprene rubber, polypropylene, polybutylene, polystyrene, polyvinyl acetate, nylon, polyester, polybutadiene, polybenzimidazole, polyacrylonitrile, epichlorohydrine, polysulfide, and polyisoprene can be cited.

In the present embodiment, such a binder can be preferably comprised in the cell adhesion layer, in the range of 5% by weight to 95% by weight, more preferably 40% by weight to 90% by weight, and particularly preferably 60% by weight to 80% by weight.

B. Base Material

Next, the base material used in the vascular cell culture substrate of this embodiment will be explained. As the base material used in this embodiment, those used as a base material for a common cell culture substrate can be used. Specifically, an inorganic material such as a glass, a metal, and a silicon, and an organic material represented by a plastic, or the like can be used.

Moreover, in this embodiment, the above-mentioned base material may have a light-shielding portion in the same pattern as the cell adhesive portion. Thereby, by the irradiation with energy from the base material side after disposing the photocatalyst-containing layer side substrate to be described later, and the above-mentioned cell adhesion layer facing with each other, the cell adhesion-inhibiting properties can be provided only in the region which is without formation of the light-shielding portion and without the adhesive properties change with the cell in the region where the light-shielding portion is formed. The light-shielding portion is not particularly limited as long as it can shield the energy to be directed at the time of forming the cell adhesion-inhibiting portion to be described later, and it can be same as the commonly used light-shielding portion, and thus the detailed description thereof is omitted herein.

C. Photocatalyst-Containing Layer Side Substrate

First, the photocatalyst-containing layer side substrate, comprising a photocatalyst-containing layer containing a photocatalyst, used in this embodiment is described. The photocatalyst-containing layer side substrate used in this embodiment usually comprises a photocatalyst-containing layer containing a photocatalyst and generally comprises a base body and a photocatalyst-containing layer formed on the base body. This photocatalyst-containing layer side substrate may also have, for example, photocatalyst-containing layer side light-shielding portion formed in a pattern form or a primer layer. The following will describe each of the constituents of the photocatalyst-containing layer side substrate used in this embodiment.

(i) Photocatalyst-Containing Layer

First, the photocatalyst-containing layer used in the photocatalyst-containing layer side substrate is described. The photocatalyst-containing layer used in this embodiment is not particularly limited insofar as the layer is constituted such that the photocatalyst in the photocatalyst-containing layer can cause the decomposition or denaturation of the cell adhesive material in the adjacent cell adhesion layer. The photocatalyst-containing layer may be composed of a photocatalyst and a binder or may be made of a photocatalyst only. The property of the surface thereof may be lyophilic or repellent to liquid.

The photocatalyst-containing layer used in this embodiment may be formed on the whole surface of a base body, or as shown in, for example, FIG. 3, a photocatalyst-containing layer 12 may be formed in a pattern form on a base body 11.

As the photocatalyst that can be used in the present embodiment, specifically, for instance, titanium dioxide (TiO₂), zinc oxide (ZnO), tin oxide (SnO₂), strontium titanate (SrTiO₃), tungsten oxide (WO₃), bismuth oxide (Bi₂O₃) and iron oxide (Fe₂O₃) that are known as photo-semiconductors can be cited. These can be used singularly or in combination of at least two kinds.

In the present embodiment, in particular, titanium dioxide, owing to a large band gap, chemical stability, non-toxicity, and easy availability, can be preferably used. There are two types of titanium dioxide, anatase type and rutile type, and both can be used in the present embodiment; however, the anatase type titanium dioxide is more preferable. An excitation wavelength of the anatase type titanium dioxide is 380 nm or less.

As such anatase type titanium dioxide, for instance, an anatase titania sol of hydrochloric acid deflocculation type (trade name: STS-02, manufactured by ISHIHARA SANGYO KAISHA, LTD., average particle diameter: 7 nm, and trade name: ST-KO1, manufactured by ISHIHARA SANGYO KAISHA, LTD.), and an anatase titania sol of nitric acid deflocculation type (trade name: TA-15, manufactured by NISSAN CHEMICAL INDUSTRIES, LTD., average particle diameter: 12 nm) can be cited.

The smaller is a particle diameter of the photocatalyst, the better, because a photocatalyst reaction is caused more effectively. It is preferable to use the photocatalyst with an average particle diameter of 50 nm or less, and one having an average particle diameter of 20 nm or less can be particularly preferably used.

The photocatalyst-containing layer in this embodiment may be made of a photocatalyst only as described above or may be formed from a mixture with a binder.

The photocatalyst-containing layer consisting of a photocatalyst only is advantageous in costs because the efficiency of decomposing or denaturing the cell adhesive material in the cell adhesion layer is improved to reduce the treatment time. On the other hand, use of the photocatalyst-containing layer comprising a photocatalyst and a binder is advantageous in that the photocatalyst-containing layer can be easily formed.

An example of the method for forming the photocatalyst-containing layer made only of a photocatalyst may be a vacuum film-forming method such as sputtering, CVD or vacuum vapor deposition. The formation of the photocatalyst-containing layer by the vacuum film-forming method makes it possible to render the layer a homogeneous photocatalyst-containing layer made only of a photocatalyst. Thereby, the cell adhesive material can be decomposed or denatured homogeneously. At the same time, since the layer is made only of a photocatalyst, the cell adhesive material can be decomposed or denatured more effectively, as compared with the case of using a binder.

Another example of the method for forming the photocatalyst-containing layer made only of a photocatalyst, is the following method: for example, in the case that the photocatalyst is titanium dioxide, amorphous titania is formed on the base material, and then, calcinating so as to phase-change the titania to crystalline titania. The amorphous titania used in this case can be obtained, for example, by hydrolysis or dehydration condensation of an inorganic salt of titanium, such as titanium tetrachloride or titanium sulfate, or hydrolysis or dehydration condensation of an organic titanium compound, such as tetraethoxytitanium, tetraisopropoxytitanium, tetra-n-propoxytitanium, tetrabutoxytitanium or tetramethoxytitanium, in the presence of an acid. Next, the resultant is calcinated at 400° C. to 500° C. so as to be denatured to anatase type titania, and calcinated at 600° C. to 700° C. so as to be denatured to rutile type titania.

In the case of using a binder, the binder preferably having a high bonding energy, wherein its principal skeleton is not decomposed by photoexcitation of the photocatalyst. Examples of such a binder include the organopolysiloxanes described in the above-mentioned item “Cell adhesion layer”.

In the case of using such an organopolysiloxane as the binder, the photocatalyst-containing layer can be formed by dispersing a photocatalyst, the organopolysiloxane as the binder, and optional additives if needed into a solvent to prepare a coating solution, and coating this coating solution onto the base material. The used solvent is preferably an alcoholic based organic solvent such as ethanol or isopropanol. The coating can be performed by a known coating method such as spin coating, spray coating, dip coating, roll coating, or bead coating. When the coating solution contains an ultraviolet curable component as the binder, the photocatalyst-containing layer can be formed by curing the coating solution through the irradiation of ultraviolet rays.

As the binder, an amorphous silica precursor can be used. This amorphous silica precursor is preferably a silicon compound represented by the general formula SiX₄, wherein X being halogen, methoxy group, ethoxy group, acetyl group or the like; silanol which is a hydrolyzate thereof; or polysiloxane having an average molecular weight of 3000 or less.

Specific examples thereof include such as tetraethoxysilane, tetraisopropoxysilane, tetra-n-propoxysilane, tetrabutoxysilane, and tetramethoxysilane. In this case, the photocatalyst-containing layer can be formed by dispersing the amorphous silica precursor and particles of a photocatalyst homogeneously into a non-aqueous solvent, hydrolyzing with water content in the air to form a silanol onto a base material, and then subjecting to dehydration polycondensation at room temperature. When the dehydration polycondensation of the silanol is performed at 100° C. or higher, the polymerization degree of the silanol increases so that the strength of the film surface can be improved. A single kind or two or more kinds of this binding agent may be used.

The content of the photocatalyst in the photocatalyst-containing layer can be set in the range of 5 to 60% by weight, preferably in the range of 20 to 40% by weight. The thickness of the photocatalyst-containing layer is preferably in the range of 0.05 to 10 μm.

Besides the above-mentioned photocatalyst and binder, the surfactant and so on used in the above-mentioned cell adhesion layer can be incorporated into the photocatalyst-containing layer.

(ii) Base Body

The following will describe the base body used in the photocatalyst-containing layer side substrate. Usually, the photocatalyst-containing layer side substrate comprises at least a base body and a photocatalyst-containing layer formed on the base body. In this case, the material which constitutes the base body to be used is appropriately selected depending on the direction of energy irradiation which will be detailed later, necessity of the resulting pattern-forming body to be transparency, or other factors.

The base body used in this embodiment may be a member having flexibility, such as a resin film, or may be a member having no flexibility, such as a glass substrate. This is appropriately selected depending on the method of the energy irradiation.

An anchor layer may be formed on the base body in order to improve the adhesion between the surface of the base body and the photocatalyst-containing layer. The anchor layer may be made of, for example, a silane based or titanium based coupling agent.

(iii) Photocatalyst-Containing Layer Side Light-Shielding Portion

The photocatalyst-containing layer side substrate used in this embodiment may be a photocatalyst-containing layer side substrate on which photocatalyst-containing layer side light-shielding portion is formed in a pattern. When the photocatalyst-containing layer side substrate having photocatalyst-containing layer side light-shielding portion is used in this way, at the time of irradiating energy, it is not necessary to use any photomask or to carry out drawing irradiation with a laser light. Since alignment of the photomask and the photocatalyst-containing layer side substrate is not necessary, process can be made simple. Further, since expensive device for drawing irradiation is also not necessary, it is advantageous in costs.

Such a photocatalyst-containing layer side substrate having photocatalyst-containing layer side light-shielding portion can be classified into the following two embodiments, depending on the position where the photocatalyst-containing layer side light-shielding portion is formed.

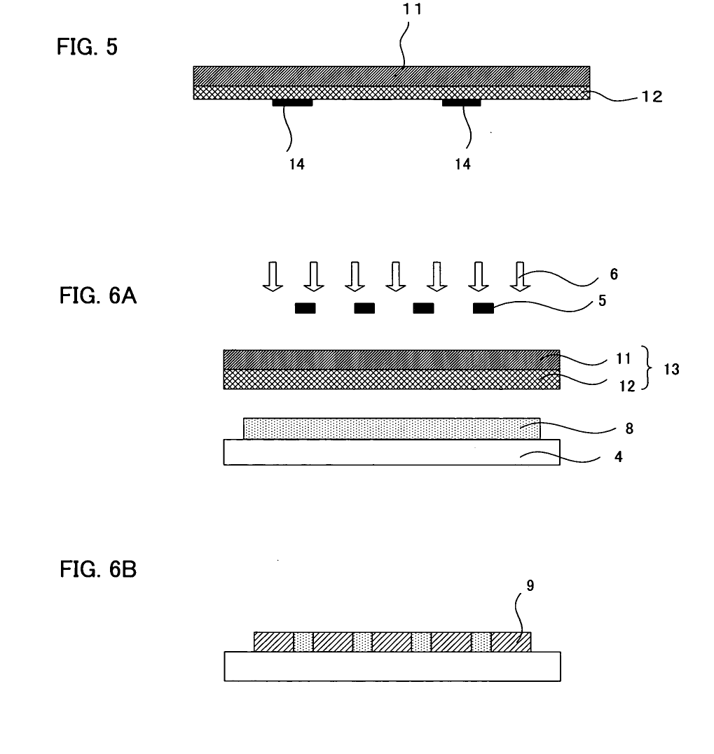

One of them is an embodiment, as shown in FIG. 4 for example, wherein photocatalyst-containing layer side light-shielding portion 14 is formed on a base body 11, and a photocatalyst-containing layer 12 is formed on the photocatalyst-containing layer side light-shielding portion 14 to obtain the photocatalyst-containing layer side substrate. The other example is an embodiment, as shown in FIG. 5 for example, wherein a photocatalyst-containing layer 12 is formed on a base body 11, and photocatalyst-containing layer side light-shielding portion 14 is formed thereon to obtain the photocatalyst-containing layer side substrate.

In any one of the embodiments, since the photocatalyst-containing layer side light-shielding portion is arranged near the region where the photocatalyst-containing layer and the cell adhesion layer are arranged, the effect of energy-scattering in the base body or the like can be made smaller than in the case of using a photomask. Accordingly, irradiation of energy in a pattern can be more precisely attained.

In this embodiment, in the case of the embodiment wherein the photocatalyst-containing layer side light-shielding portion 14 is formed on a photocatalyst-containing layer 12 as shown in FIG. 5, there is an advantage that at the time of arranging the photocatalyst-containing layer and the cell adhesion layer in a predetermined position, the photocatalyst-containing layer side light-shielding portion can be used as a spacer for making the interval constant, by making the film thickness of the photocatalyst-containing layer side light-shielding portion consistent with the width of the interval between the two layers.

In other words, when the photocatalyst-containing layer and the cell adhesion layer are arranged so as to be facing each other at a predetermined interval, by arranging the photocatalyst-containing layer side light-shielding portion and the cell adhesion layer in close contact to each other, the dimension of the predetermined interval can be made precise. When energy is irradiated in this state, cell adhesion-inhibiting portion can be formed with a good precision since cell adhesive material in the cell adhesion layer, inside the region where the cell adhesion layer and the photocatalyst-containing layer side light-shielding portion are in contact, is not decomposed or denatured.

The method for forming such photocatalyst-containing layer side light-shielding portion is not particularly limited, and may be appropriately selected in accordance with the property of the surface on which the photocatalyst-containing layer side light-shielding portion is to be formed, shielding ability against the required energy, and others. The light-shielding portion may be the same as those generally used. Thus, the detailed description thereof is omitted herein.

The above has described two cases, wherein the photocatalyst-containing layer side light-shielding portion is formed in between the base body and the photocatalyst-containing layer and is formed on the surface of the photocatalyst-containing layer. Besides, the photocatalyst-containing layer side light-shielding portion may be formed on the base body surface of the side on which the photocatalyst-containing layer is not formed. In this embodiment, for example, a photomask can be made in close contact to this surface to such a degree that the photomask is removable. Thus, this embodiment can be preferably used for the case that the pattern of the cell adhesion-inhibiting portions is changed for every small lot.

(iv) Primer Layer

The following will describe a primer layer used in the photocatalyst-containing layer side substrate of this embodiment. In this embodiment, when photocatalyst-containing layer side light-shielding portion is formed into a pattern on a base body and a photocatalyst-containing layer is formed thereon so as to prepare a photocatalyst-containing layer side substrate described above, a primer layer may be formed between the photocatalyst-containing layer side light-shielding portion and the photocatalyst-containing layer.

The effect and function of this primer layer are not necessarily clear, but would be as follows: by forming the primer layer between the photocatalyst-containing layer side light-shielding portion and the photocatalyst-containing layer, the primer layer is assumed to exhibit a function of preventing the diffusion of impurities from the photocatalyst-containing layer side light-shielding portion and openings present between the photocatalyst-containing layer side light-shielding portions, in particular, residues generated when the photocatalyst-containing layer side light-shielding portion is patterned, or metal or metal ion impurities; the impurities being factors for blocking the decomposition or denaturation of the cell adhesive material by action of the photocatalyst. Accordingly, by forming the primer layer, it is possible to process the decomposition or denaturation of the cell adhesive material with high sensitivity, so as to yield cell adhesion-inhibiting portion which are highly precisely formed.

The primer layer in this embodiment is a layer for preventing the effect of the photocatalyst from being affected by the impurities present in not only the photocatalyst-containing layer side light-shielding portion but also in the openings formed between the photocatalyst-containing layer side light-shielding portions. It is therefore preferred to form the primer layer over the entire surface of the photocatalyst-containing layer side light-shielding portion including the openings.

The primer layer in this embodiment is not particularly limited insofar as the primer layer is formed not to bring the photocatalyst-containing layer side light-shielding portion and the photocatalyst-containing layer of the photocatalyst-containing layer side substrate into contact with each other.

A material that forms the primer layer, though not particularly limited, is preferably an inorganic material that is not likely to be decomposed by the action of the photocatalyst. Specifically, amorphous silica can be cited. When such amorphous silica is used, a precursor of the amorphous silica is preferably a silicon compound that is represented by a general formula, SiX₄, wherein X being halogen, methoxy group, ethoxy group, acetyl group or the like; silanol that is a hydrolysate thereof, or polysiloxane having an average molecular weight of 3000 or less.

A film thickness of the primer layer is preferably in the range of 0.001 μm to 1 μm and particularly preferably in the range of 0.001 μm to 0.1 μm.

D. Method for Forming Cell Adhesion-Inhibiting Portion

Hereinafter, the method for forming the cell adhesion-inhibiting portion in this embodiment is described. In this embodiment, for example as shown in FIG. 6, a cell adhesion layer 8 formed on a base material 4, and a photocatalyst-containing layer 12 of a photocatalyst-containing layer side substrate 13, are arranged with a predetermined space and irradiated with energy 6 from a predetermined direction, for example, via photomask 5 (FIG. 6A). The cell adhesive material in the region irradiated with energy is thereby decomposed or denatured, thus forming the cell adhesion-inhibiting portion 9 having no adhesive properties to a blood vessel forming cell (FIG. 6B). In this case, when the cell adhesive material is decomposed for example by the action of a photocatalyst upon irradiation with energy, the cell adhesion-inhibiting portion contains a small amount of the cell adhesive material or decomposed products of the cell adhesive material. Otherwise, the cell adhesion layer is completely decomposed and removed to expose the base material or the like. When the cell adhesive material is denatured by the action of a photocatalyst upon irradiation with energy, its denatured products are contained in the cell adhesion-inhibiting portion.

The above-mentioned wording “arranging” means that the layers are arranged in the state that the action of the photocatalyst can substantially work to the surface of the cell adhesion layer, and include not only the state that the two layers actually contact each other, but also the state that the photocatalyst-containing layer and the cell adhesion layer are arranged at a predetermined interval. The dimension of the interval is preferably 200 μm or less.

In this embodiment, the dimension of the above-mentioned interval is more preferably in the range of 0.2 μm to 10 μm, even more preferably in the range of 1 μm to 5 μm, since the precision of the pattern to be obtained becomes very good and further the sensitivity of the photocatalyst becomes high so as to make good efficiency of the decomposition or denaturation of the cell adhesive material in the cell adhesion layer. This range of the interval dimension is particularly effective for the cell adhesion layer which is small in area, wherein the interval dimension can be controlled with a high precision.

Meanwhile, in the case of treating the cell adhesion layer having large are a, for example, 300 mm×300 mm or more in size, it is very difficult to make a fine interval as described above between the photocatalyst-containing layer side substrate and the cell adhesion layer without contacting each other. Accordingly, when the cell adhesion layer has a relatively large area, the interval dimension is preferably in the range of 10 to 100 μm, more preferably in the range of 50 to 75 μm. By setting the interval dimension in the above range, the following problems will not occur that: deterioration of patterning precision, such as blurring of the pattern; or the sensitivity of the photocatalyst deteriorates so that the efficiency of decomposing or denaturing the cell adhesive material is also deteriorated. Further, there is an advantageous effect that the cell adhesive material is not unevenly decomposed or denatured.

When energy is irradiated onto the cell adhesion layer having a relatively large are a as described above, the dimension of the interval, in a unit for positioning the photocatalyst-containing layer side substrate and the cell adhesion layer inside the energy irradiating device, is preferably set in the range of 10 μm to 200 μm, more preferably in the range of 25 μm to 75 μm. The setting of the interval dimension value into this range makes it possible to arrange the photocatalyst-containing layer side substrate and the cell adhesion layer without causing a large deterioration of patterning precision or of sensitivity of the photocatalyst, or bringing the substrate and the layer into contact with each other.

When the photocatalyst-containing layer and the surface of the cell adhesion layer are arranged at a predetermined interval as described above, active oxygen species generated from oxygen and water by action of the photocatalyst can easily be released. In other words, if the interval between the photocatalyst-containing layer and the cell adhesion layer is made narrower than the above-mentioned range, the active oxygen species are not easily released, so as to make the rate for decomposing or denaturing the cell adhesive material unfavorably small. If the two layers are arranged at an interval larger than the above-mentioned range, the generated active oxygen species do not reach the cell adhesion layer easily. In this case also, the rate for decomposing or denaturing the cell adhesive material may become unfavorably small.

The method for arranging the photocatalyst-containing layer and the cell adhesion layer to make such a very small interval evenly therebetween is, for example, a method of using spacers. The use of the spacers in this way makes it possible to make an even interval. At the same time, the action of the photocatalyst does not work onto the surface of the cell adhesion layer in the regions which the spacers contact. Therefore, when the spacers are rendered to have a pattern similar to that of the cell adhesion portions, the cell adhesive material only inside regions where no spacers are formed can be decomposed or denatured so that highly precise cell adhesion-inhibiting portions can be formed. The use of the spacers also makes it possible that the active oxygen species generated by action of the photocatalyst reach the surface of the cell adhesion layer, without diffusing, at a high concentration. Accordingly, highly precise cell adhesion-inhibiting portion can be effectively formed.

In this embodiment, it is sufficient that such an arrangement state of the photocatalyst-containing layer side substrate is maintained only during the irradiation of energy.

The energy irradiation (exposure) mentioned in this embodiment is a concept that includes all energy ray irradiation that can decompose or denature the cell adhesive material by the action of the photocatalyst upon irradiation with energy, and is not limited to light irradiation.

Normally, in such energy irradiation, ultraviolet light of 400 nm or less is used. This is because, as mentioned above, the photocatalyst that is preferably used as a photocatalyst is titanium dioxide, and as energy that activates a photocatalyst action by the titanium oxide, light having the above-mentioned wavelength is preferable.

As a light source that can be used in such energy irradiation, a mercury lamp, metal halide lamp, xenon lamp, excimer lamp and other various kinds of light sources can be cited.

Other than the method in which pattern irradiation is carried out via a photomask by using the above-mentioned light source, a method of carrying out drawing irradiation in a pattern by using laser such as excimer or YAG can be applied. Furthermore, as mentioned above, when the base material has the light-shielding portion in a pattern same as that of the cell adhesion portion, energy can be irradiated over the entire surface from the base material side. In this case, there are advantages in that there are no needs of the photomask and the like and a process of positional alignment and the like are also not necessary.

An amount of irradiation of energy at the energy irradiation is an amount of irradiation necessary for decomposing or denaturing the cell adhesive material by the action of the photocatalyst.

At this time, by irradiating a layer containing the photocatalyst, with energy, while heating, the sensitivity can be raised; accordingly, it is preferable in that the cell adhesive material can be efficiently decomposed or denatured. Specifically, it is preferable to heat in the range of 30° C. to 80° C.

The energy irradiation that is carried out via a photomask in this embodiment, when the above-mentioned base material is transparent, may be carried out from either direction of the base material side or a photocatalyst-containing layer side substrate. On the other hand, when the base material is opaque, it is necessary to irradiate energy from a photocatalyst-containing layer side substrate.

### (2) Second Embodiment

In the second embodiment, at least a cell adhesion-inhibiting layer, which inhibits adhesion the to blood vessel forming cell and contains a cell adhesion-inhibiting material decomposed or denatured by the action of a photocatalyst upon irradiation with energy, is formed on the base material, and in the above cell adhesion portion, the cell adhesion-inhibiting material is decomposed or denatured by the action of a photocatalyst upon irradiation with energy.

In this embodiment, the cell adhesion-inhibiting material decomposed or denatured by the action of a photocatalyst upon irradiation with energy is contained in the cell adhesion-inhibiting layer. Therefore, by arranging the cell adhesion-inhibiting layer and the photocatalyst-containing layer so as to be opposite to each other and irradiating with energy in the pattern of the cell adhesion portion, the cell adhesion-inhibiting material in the cell adhesion-inhibiting layer can be decomposed or denatured by the action of the photocatalyst in the photocatalyst-containing layer to form a cell adhesion portion having adhesive properties to a blood vessel forming cell. Because the cell adhesion-inhibiting material remains in the region not irradiated with energy, this region has no adhesive properties to a blood vessel forming cell and can be used as a cell adhesion-inhibiting portion.

The phrase “the cell adhesion-inhibiting material is decomposed or denatured” means that the cell adhesion-inhibiting material is not contained, or that the cell adhesion-inhibiting material is contained in a smaller amount than the amount of the cell adhesion-inhibiting material contained in the cell adhesion-inhibiting portion. For example, when the cell adhesion-inhibiting material is decomposed by the action of a photocatalyst upon irradiation with energy, the cell adhesion-inhibiting material is contained in a small amount in the cell adhesion portion, or decomposed products etc. of the cell adhesion-inhibiting material are contained, or the cell adhesion-inhibiting material is completely decomposed to expose the base material. When the cell adhesion-inhibiting material is denatured by the action of a photocatalyst upon irradiation with energy, its denatured products etc. are contained in the cell adhesion portion. In this embodiment, the cell adhesion portion preferably contains the cell adhesive material having adhesive properties to a blood vessel forming cell, at least after irradiation with energy. The adhesive properties to a blood vessel forming cell of the cell adhesion portion can thereby be further increased, and the blood vessel forming cell can adhere highly accurately to the cell adhesion portion only.

The surface distance of the cell adhesion-inhibiting portion in this embodiment is usually about 200 μm to 1000 μm, particularly about 300 μm to 500 μm. Blood vessel forming cells can thereby be prevented from contacting with one another via pseudopods between the adjacent cell adhesion portions.

It is also preferable in the present embodiment that the cell adhesion auxiliary portion is formed in the cell adhesion portion.

The base material, photocatalyst-containing layer side substrate, its arrangement, the energy irradiation method, the shape of the cell adhesion portion, the cell adhesion auxiliary portion etc. used in this embodiment are the same as those described in the first embodiment described above, and thus their detailed description is omitted herein. Hereinafter, the cell adhesion-inhibiting layer used in this embodiment is described.

The cell adhesion-inhibiting layer used in this embodiment is not particularly limited insofar as it has cell adhesion-inhibiting properties of inhibiting adhesion to the blood vessel forming cell and contains a cell adhesion-inhibiting material to be decomposed or denatured by the action of a photocatalyst upon irradiation with energy.

In this embodiment, the method for forming the layer and the like is not particularly limited insofar as such layer can be formed. For example, the layer can be formed by coating a cell adhesion-inhibiting layer-forming coating solution containing the cell adhesion-inhibiting material, onto the photocatalyst-containing layer, by a common coating method. The thickness of the cell adhesion-inhibiting layer can be suitably selected depending on the type etc. of the vascular cell culture substrate, and can usually be about 0.01 μm to 1.0 μm, particularly about 0.1 μm to 0.3 μm.

The type etc. of the cell adhesion-inhibiting material used in this embodiment are not particularly limited insofar as the cell adhesion-inhibiting material has cell adhesion-inhibiting properties of inhibiting adhesion to the blood vessel forming cell and is decomposed or denatured by the action of a photocatalyst upon irradiation with energy.

The phrase “to have cell adhesion-inhibiting properties” means to have a property of preventing the blood vessel forming cell from being adhered to the cell adhesion-inhibiting material, and when the adhesive properties to a blood vessel forming cell varies depending on the type of the blood vessel forming cell, the phrase means to have a property of inhibiting adhesion with the objective blood vessel forming cell.

The cell adhesion-inhibiting material used in this embodiment is a material having such cell adhesion-inhibiting properties. A material, which loses the cell adhesion-inhibiting properties or which obtains good adhesive properties to a blood vessel forming cell, when decomposed or denatured by the action of a photocatalyst upon irradiation with energy is used.

As the cell adhesion-inhibiting material, a material having high hydration ability can be used as an example. The material having high hydration ability forms a hydration layer wherein water molecules gather around thereof. Usually, since such a material having high hydration ability has higher adhesion to water molecules than adhesion to the blood vessel forming cell, the blood vessel forming cell cannot be adhered to the material having high hydration ability. Thus, the layer will have low adhesive properties to a blood vessel forming cell. The hydration ability is referred to as a property of hydrating with water molecules, and high hydration ability is intended to mean that the material is easily hydrated with water molecules.

As the material having high hydration ability which is used as a cell adhesion-inhibiting material, for example, polyethylene glycol, amphoteric ionic materials having a betaine structure, or phospholipid-containing materials can be listed. When such materials are used as the cell adhesion-inhibiting material, upon irradiated with energy in the below-described energy irradiating process, the cell adhesion-inhibiting material is decomposed or denatured by the action of a photocatalyst so as to remove the hydration layer on the surface, thereby obtaining the material not having the cell adhesion-inhibiting properties.

In this embodiment, a surfactant, which is decomposed by the action of a photocatalyst and has water repellent or oil repellent organic substituent, can also be used as the cell adhesion-inhibiting material. As such surfactant for example, nonionic surfactants such as: hydrocarbon based such as the respective series of NIKKOL BL, BC, BO, and BB manufactured by Nikko Chemicals Co., Ltd.; and fluorine based or silicone based such as ZONYL FSN and FSO manufacture by Du Pont Kabushiki Kaisha, Surflon S-141 and 145 manufactured by ASAHI GLASS CO., LTD., Megaface F-141 and 144 manufactured by DAINIPPON INK AND CHEMICALS, Inc., FTERGENT F-200 and F251 manufactured by Neos, UNIDYNE DS-401 and 402 manufactured by DAIKIN INDUSTRIES, Ltd., and Fluorad FC-170 and 176 manufactured by 3M can be cited. Also, cationic surfactants, anionic surfactants and amphoteric surfactants also can be used.

When the cell adhesion-inhibiting layer is formed by using the above material as the cell adhesion-inhibiting material, the cell adhesion-inhibiting material is unevenly distributed on the surface. The water repellency or oil repellency on the surface can thereby be increased, and the interaction with the blood vessel forming cell can be decreased to reduce adhesive properties to a blood vessel forming cell. Upon irradiation of this layer with energy in the energy irradiating process, the material is easily decomposed by the action of the photocatalyst to expose the photocatalyst. Thus, one not having the cell adhesion-inhibiting properties can be obtained.

In this embodiment, a material, which obtains good adhesive properties to a blood vessel forming cell by the action of the photocatalyst upon irradiation with energy, is particularly preferably used as the cell adhesion-inhibiting material. As such cell adhesion-inhibiting material, for example, materials having oil repellency or water repellency can be listed.

When the material having oil repellency or water repellency is used as the cell adhesion-inhibiting material, the interaction such as hydrophobic interaction between the blood vessel forming cell and the cell adhesion-inhibiting material is made low by the water repellency or oil repellency of the cell adhesion-inhibiting material, thereby decreasing adhesive properties to a blood vessel forming cell.

As the material having water repellency or oil repellency, a material, for example, which has such high bonding energy that the skeleton thereof is not decomposed by the action of the photocatalyst and has water repellent or oil repellant organic substituent to be decomposed by action of the photocatalyst, can be listed.

Examples of such a material, which has such high bonding energy that the skeleton thereof is not decomposed by the action of the photocatalyst and has water repellent or oil repellant organic substituent to be decomposed by action of the photocatalyst, include, for example, the materials used as the binder in the first embodiment, that is, (1) the organopolysiloxanes exhibiting high strength, obtained by hydrolyzing or polycondensating chloro- or alkoxysilanes by sol-gel reaction etc. and (2) organopolysiloxanes obtained by crosslinking reactive silicone.

When such material is used as the binder in the first embodiment, the material is used as a material having cell adhesion-inhibiting properties by decomposing or denaturing the above-mentioned side chains of the organopolysiloxanes, in high ratio, so as to make it super hydrophilic by the action of the photocatalyst upon irradiation with energy. However, in this embodiment, the region irradiated with the energy can have adhesive properties to a blood vessel forming cell by irradiating with energy to such a degree that side chains of the organopolysiloxanes are not completely decomposed or denatured by the action of the photocatalyst upon irradiation with energy. Together with the above-mentioned organopolysiloxane, a stable organosilicon compound not undergoing any crosslinking reaction, such as dimethylpolysiloxane, can also be separately mixed.

When the material having water repellency or oil repellency is used as the cell adhesion-inhibiting material, the material preferably has a contact angle, with water, of 80° or more, particularly in the range of 100° to 130°. With this contact angle given, the adhesive properties to a blood vessel forming cell, of the cell adhesion-inhibiting layer before irradiation with energy can be reduced. The upper limit of the angle is the upper limit of the contact angle, with water, of the cell adhesion-inhibiting material on a flat base material. For example, when the contact angle, with water, of the cell adhesion-inhibiting material on a base material with concavoconvex is measured, the upper limit may be about 160° as shown by Ogawa et al. in Japanese Journal of Applied Physics, Part 2, Vol. 32, L614-L615, 1993.

When this cell adhesion-inhibiting material is irradiated with energy to impart the adhesive properties to a blood vessel forming cell, the material is preferably irradiated with energy such that the contact angle thereof with water comes to be in the range of 10° to 40°, particularly 15° to 30°. The adhesive properties to a blood vessel forming cell of the cell adhesion-inhibiting layer after energy irradiation can thereby be increased. The contact angle with water can be obtained by the method described above.

The cell adhesion-inhibiting material is contained preferably in the range of 0.01% by weight to 95% by weight, particularly 1% by weight to 10% by weight, in the cell adhesion-inhibiting layer. The region containing the cell adhesion-inhibiting material can thereby be a region of low adhesive properties to a blood vessel forming cell.

The cell adhesion-inhibiting material preferably has surface activity. For example, when drying the cell adhesion-inhibiting layer-forming coating solution or the like containing the cell adhesion-inhibiting material after coating thereof, the material is distributed highly unevenly on the surface of the coating film, thus giving excellent cell adhesion-inhibiting properties.

The cell adhesion-inhibiting layer in this embodiment may contain a binder and the like in accordance with required characteristics such as coating properties in formation of the layer, strength and resistance of the formed layer. The cell adhesion-inhibiting material may also function as the binder.

As the binder, for example, a binder having such high bonding energy that its principal skeleton is not decomposed by the action of the photocatalyst can be used. Specific examples of the binder include such as polysiloxane not having organic substituents or having organic substituents to such a degree that adhesive properties are not adversely affected, and such polysiloxane can be obtained by hydrolyzing or polycondensating such as tetramethoxysilane or tetraethoxysilane.

In this embodiment, the binder is contained preferably in the range of 5% by weight to 95% by weight, more preferably 40% by weight to 90% by weight, still more preferably 60% by weight to 80% by weight, in the cell adhesion-inhibiting layer. By incorporation of the binder in this range, formation of the cell adhesion-inhibiting layer can be facilitated and the cell adhesion-inhibiting layer can be endowed with strength etc. thus allowing it to exhibit its characteristics.

In this embodiment, the cell adhesion-inhibiting layer preferably contains a cell adhesive material having adhesive properties to a blood vessel forming cell, at least after irradiation with energy. By this, in the cell adhesion-inhibiting layer, adhesive properties to a blood vessel forming cell of the cell adhesion portion, which is the region irradiated with energy, can be further improved. The cell adhesive material may be a material usable as the binder or may be a material used separately from the binder. The cell adhesive material may have good adhesive properties to a blood vessel forming cell prior to irradiation with energy, or may be endowed with good adhesive properties to a blood vessel forming cell by the action of the photocatalyst upon irradiation with energy. The wording “having adhesive properties to a blood vessel forming cell” refers to good adhesion to the blood vessel forming cell, and when the adhesive properties to a blood vessel forming cell vary depending on the type of the blood vessel forming cell, the wording refers to good adhesion to the target blood vessel forming cell.

In this embodiment, as long as the cell adhesive material have good adhesive properties to a blood vessel forming cell at least after being irradiated with energy, the adhesive properties to a blood vessel forming cell can be improved, for example, by biological characteristics or by physical interaction such as hydrophobic interaction, electrostatic interaction, hydrogen bonding, van der Waals force.

In this embodiment, the cell adhesive material is contained preferably in the range of 0.01% by weight to 95% by weight, particularly 1% by weight to 10% by weight, in the cell adhesion-inhibiting layer. By this, the cell adhesion-inhibiting layer can further improve the adhesive properties to a blood vessel forming cell of the cell adhesion portion, which is a region irradiated with energy. When the material having good adhesive properties to a blood vessel forming cell prior to irradiation with energy is used as the cell adhesive material, the material is preferably contained to such a degree as not to inhibit the cell adhesion-inhibiting properties of the cell adhesion-inhibiting material in the region not irradiated with energy, that is, the region serving as the cell adhesion-inhibiting portion.

### (3) OTHERS

The present invention is not limited to the above-mentioned two embodiments, and for example, the vascular cell culture substrate with the above-mentioned cell adhesive portion and the above-mentioned cell adhesion-inhibiting portion formed may be provided by forming a photocatalyst-containing layer containing at least a photocatalyst on a base material, forming the cell adhesion layer or the cell adhesion-inhibiting layer thereon, and carrying out the irradiation with energy. Moreover, the vascular cell culture substrate with the above-mentioned cell adhesive portion and the above-mentioned cell adhesion-inhibiting portion formed may be provided by for example forming a layer with the cell adhesive material or the cell adhesion-inhibiting material mixed with a photocatalyst, and directing the energy to the layer. Since the photocatalyst, the cell adhesion layer, the cell adhesion-inhibiting layer, the cell adhesive material, the cell adhesion-inhibiting material, or the like used in the vascular cell culture substrate are same as those explained in the above-mentioned two embodiments, the detailed description thereof is omitted herein.

<Cell>

Next, the cell used in the present invention will be explained. The cell used in the present invention is not particularly limited as long as it is activated by the supply of such as the oxygen or nutrition from the above-mentioned blood vessels so as to provide an artificial tissue. For example, cell species having a metabolism such as a hepatocyte and a Langerhans Island cell, or cell species of an information transmitting system, such as a brain cell and a nerve cell can be presented. The above-mentioned cells used for the above-mentioned blood vessel-containing tissue layer is not limited to one kind, but plural kinds of cells can be used in combination.

As the method for disposing the cell between the above-mentioned blood vessels, as mentioned above, a method of providing a tissue by for example disposing the blood vessels on such as a culture medium with the distance between the adjacent blood vessels as the above-mentioned nutrition supplyable distance, and seeding the cell on the culture medium between the blood vessels and culturing can be presented. Moreover, a method of culturing the above-mentioned cell on a culture medium independently from the blood vessels for providing such as a tissue like a sheet, and disposing the same on the blood vessels disposed with the nutrition supplyable distance can also be used.

The culture medium or the like for culturing the above-mentioned cell can be selected optionally according to the purposed cell so that one used for culture of a common cell can be used, and thus the detailed description thereof is omitted herein.

2. Artificial Tissue

Next, the artificial tissue of the present invention will be explained. The artificial tissue of the present invention is not particularly limited as long as it has the above-mentioned blood vessel-containing tissue layer so that the blood vessel-containing tissue layer may be provided as only one layer or as a lamination of two or more layers. By laminating by two or more layers, the above-mentioned blood vessels and cell can be disposed three-dimensionally so that an artificial tissue of a more complicated structure can be provided.

In the case the blood vessel-containing tissue layer is laminated, the number of the laminated layers differs significantly depending on such as the kind of the purposed artificial tissue or the size, however, it is in general about 2 to 100 layers, and in particular, about 2 to 10 layers.

The above-mentioned artificial tissue in the present invention may optionally comprise other members as needed in addition to the above-mentioned blood vessels and cells.

Here, the artificial tissue of the present invention may be, for example, an artificial liver, an artificial pancreas, an artificial nerve circuit, or an artificial retina.

B. Process for Producing Artificial Tissue

Next, the process for producing an artificial tissue of the present invention will be explained. The process for producing an artificial tissue of the present invention is a process for producing an artificial tissue comprising a blood vessel-containing tissue layer having at least two adjacent blood vessels and a cell disposed between the blood vessels, wherein the process comprising:

a blood vessel disposing process of disposing two adjacent blood vessels with a nutrition supplyable distance which does not cause a necrosis of the cell, and

a cell contacting process of contacting a cell containing layer containing the cell and the blood vessels. Here, the above-mentioned nutrition supplyable distance is same as the nutrition supplyable distance explained in the above-mentioned “A. Artificial tissue”.

According to the present invention, since the blood vessels are disposed by the nutrition supplyable distance in the blood vessel disposing process, the nutrition can be supplied to the cell contacted by the cell contacting process through the blood vessels. Therefore, the cell cannot be perished in the formed artificial tissue, or the like so that various artificial tissues to be used as, for example, an organ can be provided.

Hereinafter, each process of the process for producing an artificial tissue of the present invention will be explained.

1. Blood Vessel Disposing Process

First, the blood vessel disposing process of the process for producing an artificial tissue of the present invention will be explained. The blood vessel disposing process in the present invention is a process of disposing at least two adjacent blood vessels with the nutrition supplyable distance which does not cause a necrosis of the above-mentioned cell.

Here, as to the method for disposing the blood vessels, as long as the blood vessels can be formed such that the distance between the adjacent two blood vessels on the same substrate can be the nutrition supplyable distance, the blood vessels formed on the substrate can be used as they are. However, in general, as mentioned above, it is difficult to form adjacent blood vessels by a close distance. Therefore, it is preferable that the process is a process of forming at least two blood vessels with an interval of the nutrition supplyable distance or more, and thereafter disposing the same by the nutrition supplyable distance.

As the method for disposing the blood vessels by such a distance, for example, a method of forming the blood vessels on the vascular cell culture substrate with the interval of more than the nutrition supplyable distance, detaching the formed blood vessels form the vascular cell culture substrate, and disposing the same by the nutrition supplyable distance can be presented.

Moreover, for example a method of forming the blood vessels on the vascular cell culture substrate with an interval of more than the nutrition supplyable distance, and removing a part of the vascular cell culture substrate between the adjacent blood vessels can also be presented. In this case, for example, a method of partially cutting the vascular cell culture substrate after formation of the blood vessels may be used, however, it can also be used a method of, for example, forming the above-mentioned blood vessels on the vascular cell culture substrate comprising a plurality of plates, and after formation of the blood vessels, detaching the plates between the adjacent blood vessels.

Furthermore, it can also be used a method of stretching the vascular cell culture substrate having the stretching properties, forming on the vascular cell culture substrate with an interval of more than the nutrition supplyable distance on the vascular cell culture substrate, and then shortening the vascular cell culture substrate so as to dispose the blood vessels by the nutrition supplyable distance. At the time, as the vascular cell culture substrate to be used, for example, a silicone rubber, or a surface process product thereof can be presented.

Moreover, in the present invention, it is also possible to execute the blood vessel disposing process after the cell contacting process to be described later. In this case, for example, a cell contacting process of forming at least two blood cells on the vascular cell culture substrate with an interval of the nutrition supplyable distance or more, and contacting the formed blood vessels and cell is executed. Thereafter, by supplying the blood and the like in the blood vessels and removing the cell in the portion with the cell perished without the supply of the nutrition and oxygen from the blood vessels between the above-mentioned blood vessels, the blood vessels can be disposed by the nutrition supplyable distance.

As the method for forming the blood vessels, as mentioned above, it is preferable to use a method of forming on a base material a cell adhesion layer containing a cell adhesive material having the adhesive properties with a cell, to be decomposed or denatured by the function of a photocatalyst accompanied by the irradiation with energy, or a cell adhesion-inhibiting layer containing a cell adhesion-inhibiting material having the cell adhesion-inhibiting properties with a cell, to be decomposed or denatured by the function of a photocatalyst accompanied by the irradiation with energy; and providing the function of the photocatalyst accompanied by the irradiation with energy in a pattern so as to provide the cell adhesive properties only in the pattern for culturing blood vessel forming cells. According to the method, since the region other than the region for culturing the blood vessel forming cells can have the cell adhesion-inhibiting properties, the blood vessel forming cells can be formed easily in a purposed pattern. Furthermore, since the morphological change, or the like of the cell for forming a tissue by the stimuli received by the blood vessel forming cells can easily be generated between the region having the cell adhesive properties and the region having the cell adhesion-inhibiting properties so that the blood vessels can be formed easily. In this case, the substrate having the cell adhesion layer or the cell adhesion-inhibiting layer can be used as the vascular cell culture substrate.

Since the material of the blood vessels, the vascular cell culture substrate used in this process, or the like, are same as those explained in the item of the blood vessel of the above-mentioned “A. Artificial tissue”, the detailed description thereof is omitted herein.

2. Cell Contacting Process

Next, the cell contacting process of the present invention will be explained. The cell contacting process of the present invention is a process of contacting a cell containing layer containing the above-mentioned cell, and the above-mentioned blood vessels.

As such a method for contacting a cell with the blood vessels, for example a method of disposing the blood vessel on a culture medium capable of culturing a cell such that the distance between the adjacent blood vessels can be the above-mentioned nutrition supplyable distance, and then disseminating and culturing a cell on the culture medium between the blood vessels can be presented. At the time, the culture medium with the blood vessels formed can be used as it is. Moreover, in the case the blood vessels are formed by forming a region having preferable adhesive properties with a cell by providing the function of a photocatalyst accompanied by the irradiation with energy to the cell adhesion-inhibiting layer having the adhesion-inhibiting properties with a cell, and utilizing the adhesive properties difference with the cell of the surface, the cell may be cultured by providing the function of a photocatalyst accompanied by the irradiation with energy again to the cell adhesion-inhibiting layer at the time of disseminating the cell for providing preferable adhesive properties with the cell of the region between the blood vessels.

Moreover, it is also possible that the above-mentioned cell is cultured on a culture medium, or the like independently from the blood vessels so as to have a sheet-like cell tissue, and dispose the same on the above-mentioned blood vessels disposed by the nutrition supplyable distance for contacting the blood vessels and the cell. Moreover, it is also possible to contact the cells with the d blood vessels on the above-mentioned sheet-like cells, or the like disposed so as to have the distance between the adjacent blood vessels as the nutrition supplyable distance. In this case, the above-mentioned cell contacting process and the above-mentioned blood vessel disposing process are carried out at the same time. Here, since the cell, or the like used in this process are same as those explained in the item of the cell of the above-mentioned “A. Artificial tissue”, the detailed description thereof is omitted herein.

3. Others

In the present invention, a necessary process such as a process of laminating the blood vessel-containing tissue formed by carrying out the above-mentioned blood vessel disposing process and cell contacting process may optionally be included as needed.

The present invention is not limited to the above-mentioned embodiments. The above-mentioned embodiments are examples, and any one having the substantially same configuration as the technical idea mentioned in the claims of the present invention for providing the same effects is incorporated in the technical range of the present invention.

### EXAMPLES

Hereinafter, the present invention will be explained further specifically with reference to the examples.

### Example 1

(Formation of a Vascular Cell Culture Substrate Having a Light-Shielding Layer)

A quartz photo mask having a stripe pattern of 40 μm of a glass portion as the cell adhesive portion, and 300 μm of a metal light-shielding portion as the cell adhesion-inhibiting portion was produced.

Then, 30 g of isopropyl alcohol, 4 g of trimethoxymethylsilane TSL8114 (GE Toshiba Silicones), 1 g of fluoroalkylsilane TSL-8233 (Toshiba Silicones) and 15 g of a photocatalyst inorganic coating agent ST-K03 (ISHIHARA SANGYO KAISYA, LTD.) were mixed and stirred at 100° C. for 20 minutes. The mixture was diluted 10-fold with isopropyl alcohol to prepare a photocatalyst-containing vascular cell adhesion layer composition.

A vascular cell culture substrate having a transparent photocatalyst containing vascular cell adhesion layer comprising a photocatalyst was formed by applying above-mentioned photocatalyst-containing vascular cell adhesion layer composition onto the rear side of the light-shielding layer of the photo mask substrate by a spin coater, and carrying out a drying process at 150° C. for 10 minutes.

(Patterning of the Substrate)

A vascular cell patterning culture substrate having the cell adhesive properties surface patterned with the cell adhesion-inhibiting properties in the unexposed portion and the cell adhesive properties in the exposed portion was obtained by carrying out the ultraviolet ray exposure with a mercury lamp by an energy amount of 6 J/cm² from the light-shielding layer surface side of the vascular cell culture substrate.

(Dissemination of Vascular Cells and Formation of Tissue)

The substrate was dipped in DMEM medium containing 10% bovine fetal serum, and rat vein endothelial cells were disseminated. The vascular cells were cultured at 37° C. in a 5% carbon dioxide atmosphere for 24 hours to allow the vascular cells to adhere to the cell adhesion portion.

When the vascular cells that had adhered to the substrate were observed, it was confirmed that the vascular cells were aligned along all region in the cell adhesion region, the vascular cells were in an extended form, and there is no contacting of the pseudopods between the cell adhesion portions. Further, the DMEM medium was exchanged with one containing bFGF (Sigma) at a concentration of 10 ng/ml, culturing was continued at 37° C. in a 5% carbon dioxide atmosphere for 24 hours, and formation of a regenerated vascular tissue composed of continuous vascular cells was confirmed.

(Evaluation of the Tissue)

With a collagen type I sponge (produced by Nippon Meat Packers, Inc.) swelled preliminarily in a culture medium, a rat hepatocyte cell was disseminated and cultured for 24 hours for fixing the hepatocyte cell on the sponge. With the upper and lower surfaces of the hepatocyte cell disseminated sponge contacted with the regenerative blood vessel surface of the patterning substrate for a vascular cell culture having the above-mentioned regenerative blood vessels, it was sealed in a resin container. By circulating one hour a culture medium with the hydrogen partial pressure adjusted in the regenerative blood vessels with respect to the sealed cell tissues and releasing the sealed state for observing the hepatocyte cells, the existence of the cells was confirmed.

### Comparative Example 1

An experiment was carried out in the same manner as in Example 1 except that the photomask was exchanged to one having a stripe pattern with 40 μm cell adhesion portions/100 μm cell adhesion-inhibiting portions. As a result, extinction of the hepatocyte cells in the formed pseudo-cell tissues was confirmed.

### Comparative Example 2

In the same procedure as in the example 1 except that the photomask was changed to a stripe pattern of a 40 μm of the cell adhesive portion and 150 μm of the cell adhesion-inhibiting portion, a vascular cell culturing substrate was produced, and furthermore, a rat vein endothelial cell was disseminated by the same procedure. Although the vascular endothelial cells were patterned, formation of the pseudopods was confirmed between the adjacent lines.

Furthermore, with the DMEM culture medium changed with one having bFGF (produced by Sigma) added by a 10 ng/ml concentration, culture was continued for 24 hours in a 37° C., 5% nitrogen dioxide environment. According to the observation thereof, the adjacent regenerated vascular tissues were adhered.

### Example 2

(Formation of a Photomask Having a Photocatalyst-Containing Layer)

A quartz photo mask having a stripe pattern of 40 μm of a metal light shielding portion as the cell adhesive portion, and 1000 μm of a glass portion as the cell adhesion-inhibiting portion was produced.

Mixed and stirred for 8 hours were 5 g of trimethoxymethylsilane TSL8114 (GE Toshiba Silicones) and 2.5 g of 0.5 N hydrochloric acid. The mixture was diluted 10-fold with isopropyl alcohol to prepare a primer layer composition. This primer layer composition was coated onto the patterned surface of the photomask by spin coating, and the substrate was dried at a temperature of 150° C. for 10 minutes to form a photomask provided with a primer layer.

Then, 30 g of isopropyl alcohol, 3 g of trimethoxymethylsilane TSL8114 (GE Toshiba Silicones), and 20 g of a photocatalyst inorganic coating agent ST-K03 (ISHIHARA SANGYO KAISYA, LTD.) were mixed and stirred at 100° C. for 20 minutes. The mixture was diluted 3-fold with isopropyl alcohol to prepare a photocatalyst-containing layer composition.

This photocatalyst-containing layer composition was coated, by spin coating, onto the photomask substrate provided with the primer layer, and then, dried at 150° C. for 10 minutes to form a photomask having a transparent photocatalyst-containing layer.

(Formation of a Patterning Substrate for a Vascular Cell Culture Having a Cell Adhesion Layer)

Five (5.0) grams of organosilane TSL-8114 (GE Toshiba Silicones), 0.7 g of alkylsilane LS-5258 (Shin-Etsu Chemical Co., Ltd.) and 2.36 g of 0.005 N hydrochloric acid were mixed and stirred for 24 hours. This solution was diluted 100-fold with isopropyl alcohol and coated by spin coating onto a soda glass substrate preliminary subjected to alkali treatment, and the substrate was dried at a temperature of 150° C. for 10 minutes to allow hydrolysis and polycondensation reaction to advance to give a substrate for a vascular cell culture having a vascular cell adhesive material layer of 0.2 μm in thickness.

(Patterning of the Vascular Cell Culture Substrate)

The vascular cell adhesive material layer of the above-mentioned vascular cell culture substrate was opposed to the photocatalyst-containing layer of the above-mentioned photomask containing a photocatalyst containing layer. Then, the above was exposed via the photomask to ultraviolet rays, with 6 J/cm² energy, from a mercury lamp. Thereby, a vascular cell culture substrate having a vascular cell adhesive surface patterned, such that the exposed portions having vascular cell adhesion-inhibiting properties and the unexposed portions having vascular cell adhesive properties, was obtained.

(Disseminating and Organization of the Vascular Cells)

In the same procedure as in the example 1, a vascular cell was disseminated on the substrate. According to the observation of the vascular cells adhered on the vascular cell culture substrate, it was confirmed that the vascular cells are aligned in the direction along the entire region in the cell culture region, and furthermore, that they have a stretched shape, and that contact of the pseudopods is not present between the cell adhesive portions.

Furthermore, organization of the cells was carried out in the same procedure as in the example 1 so as to confirm the formation of a regenerated vascular tissue with the cells provided continuously.

(Partial Removal of the Vascular Cell Culture Substrate)

After removing the cell adhesion-inhibiting portion provided between a blood vessel and a blood vessel of the substrate with the blood vessels formed by a 700 μm width from the central portion of the inhibiting portion, the substrate with the blood vessel formation was re-arranged for shortening the distance between the blood vessels from 1,000 μm to 300 μm.

(Evaluation of the Tissue)

According to the same tissue evaluation experiment as in the example 1, it was confirmed that the hepatocyte cells are not perished.

### Comparative Example 3

In the same procedure as in the example 2, dissemination and organization of the cells were carried out. Next, without the removing process of the substrate space portion, the tissue was evaluated with the distance between the blood vessels on the substrate remaining 1,000 μm, and as a result, necrosis of the hepatocyte cells was confirmed.

### Example 3

(Formation of a Vascular Cell Culture Substrate Having a Light-Shielding Layer and Patterning of the Substrate)

A quartz photo mask having a stripe pattern of 70 μm of a glass portion as the cell adhesive portion, and 300 μm of a metal light-shielding portion as the cell adhesion-inhibiting portion was produced. Subsequently, in the same manner as in the example 1 except that the above-mentioned quarts photo mask was used, a vascular cell culture substrate was formed. Thereafter, patterning of the vascular cell culture substrate was carried out in the same manner as in the example 1 for obtaining a vascular cell patterning culture substrate.

(Surface Treatment of the Vascular Cell Patterning Culture Substrate)

A solution with a collagen coating type I collagen (Nitta Gelatin Inc., type I-C) diluted with a pH3 acidic solution by 20 times was prepared. The above-mentioned vascular cell patterning culture substrate was impregnated in the solution along the direction of the stripe pattern, and then slowly pulled out vertically. By the operation, lines of the collagen solution were formed only in the cell adhesive portion of the vascular cell patterning culture substrate. The collagen solution was not adhered to the other portions owing to the water repellency of the adhesion-inhibiting portion. By drying the vascular cell patterning culture substrate at the room temperature, a vascular cell patterning culture substrate with the collagen coating only in the cell adhesive portion was produced.

(Dissemination of Cells and Formation of Tissue)

The above-mentioned vascular cell patterning culture substrate was dipped in DMEM medium containing 10% bovine fetal serum, and primary human umbilical vein endothelial cells (HUVECs) were disseminated. The cells were cultured at 37° C. in a 5% carbon dioxide atmosphere for 36 hours to allow the HUVECs to adhere to the cell adhesion portion. When the HUVECs that had adhered to the substrate were observed, it was confirmed that the HUVECs were aligned along all region in the cell adhesion portion, the HUVECs were in an extended form, and there is no contacting of the pseudopods between the cell adhesion portions. Further, the DMEM medium was exchanged with one containing bFGF (Sigma) at a concentration of 10 ng/ml, culturing was continued at 37° C. in a 5% carbon dioxide atmosphere for 48 hours, and formation of a regenerated vascular tissue composed of continuous HUVECs was confirmed.

(Evaluation of the Tissue)

The same tissue evaluation experiment as in the example 1 was carried out to confirm that the hepatocyte cells are not perished.

### Example 4

(Formation of a Vascular Cell Culture Substrate Having a Light-Shielding Layer and Patterning of the Substrate)

A quartz photo mask having a stripe pattern of 150 μm of a glass portion as the cell adhesive portion, and 300 μm of a metal light-shielding portion as the cell adhesion-inhibiting portion was produced. Subsequently, in the same manner as in the example 1 except that the above-mentioned quarts photo mask was used, a vascular cell culture substrate was formed. Thereafter, patterning of the vascular cell culture substrate was carried out in the same manner as in the example 1 for obtaining a vascular cell patterning culture substrate.

(Surface Treatment of the Vascular Cell Patterning Culture Substrate)

In the same way as Example 3, a vascular cell patterning culture substrate with the collagen coating only in the cell adhesive portion was produced.

(Dissemination of Cells and Formation of Tissue)

The vascular cell patterning culture substrate was dipped in DMEM medium containing 10% bovine fetal serum, and primary human umbilical vein endothelial cells (HUVECs) were disseminated. The culture dish was disposed on a shaking machine placed in an incubator with the shaking direction coinciding with the stripe direction of the substrate. By the culture for 36 hours in a 37° C., 5% carbon dioxide environment, the HUVECs was adhered onto the cell adhesive portion. During the culture period, the culture dish was slowly shaken continuously. Under microscopic observation, it was confirmed that the HUVECs were aligned along all region in the cell adhesion portion, the HUVECs were in an extended form, and there is no contacting of the pseudopods between the cell adhesion portions. Further, the DMEM medium was exchanged with one containing bFGF (Sigma) at a concentration of 10 ng/ml, culturing was continued at 37° C. in a 5% carbon dioxide atmosphere for 48 hours, and formation of a regenerated vascular tissue composed of continuous HUVECs was confirmed.

(Evaluation of the Tissue)

The same tissue evaluation experiment as in the example 1 was carried out to confirm that the hepatocyte cells are not perished.

### Example 5

(Formation of a Vascular Cell Culture Substrate Having a Light-Shielding Layer and Patterning of the Substrate)

A quartz photo mask having a total 220 μm width stripe pattern as the cell adhesive portion of: 70 μm of a glass portion, 5 μm of a metal light-shielding portion, 70 μm of a glass portion, 5 μm of a metal light-shielding portion, 70 μm of a glass portion as the cell adhesion auxiliary portion; and a 300 μm stripe pattern of a metal light-shielding portion as the cell adhesion-inhibiting portion was produced. Subsequently, in the same manner as in the example 1 except that the above-mentioned quarts photo mask was used, a vascular cell culture substrate was formed. Thereafter, patterning of the vascular cell culture substrate was carried out in the same manner as in the example 1 for obtaining a vascular cell patterning culture substrate.

(Dissemination and Organization of the Vascular Cell)

By culturing a rat vein endothelial cell on the above-mentioned vascular cell patterning culture substrate by the same culture conditions as in the example 4, formation of a regenerated vascular tissue was confirmed. The culture time was same as that in the example 1.

(Evaluation of the Tissue)

The same tissue evaluation experiment as in the example 1 was carried out to confirm that the hepatocyte cells are not perished.

What is claimed is:

1. An artificial tissue including a blood vessel-containing tissue layer having at least two adjacent blood vessels and a cell disposed between the blood vessels,
wherein an interval between the two adjacent blood vessels in the blood vessel-containing tissue layer is formed by a nutrition supplyable distance which does not cause a necrosis of the cell.

2. The artificial tissue according to claim 1, wherein the blood vessel-containing tissue layer is laminated by at least two or more layers.

3. A process for producing an artificial tissue comprising a blood vessel-containing tissue layer having at least two adjacent blood vessels and a cell disposed between the blood vessels, wherein:
a blood vessel disposing process of disposing the two adjacent blood vessels with a nutrition supplyable distance which does not cause a necrosis of the cell, and
a cell contacting process of contacting a cell containing layer containing the cell and the blood vessels are comprised.

4. The process for producing an artificial tissue according to claim 3, wherein the blood vessel disposing process is a process of forming at least two or more of the blood vessels on a vascular cell culture substrate so that the blood vessels have a distance wider than the nutrition supplyable distance, and removing a part of the vascular cell culture substrate disposed between the blood vessels.

5. The process for producing an artificial tissue according to claim 3, wherein the blood vessel disposing process is a process of forming at least two or more of the blood vessels in a state with a vascular cell culture substrate stretched on the vascular cell culture substrate having stretching properties, and shortening the vascular cell culture substrate so as to shorten a distance between the blood vessels.
